(19) United States
(12) Patent Application Publication
(10) Pub. No.: US 2003/0008355 A1
(43) Pub. Date: Jan. 9, 2003

(54) High-affinity antibodies

(76) Inventor: Peter Harrison, Surrey (GB)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/199,908

(22) Filed: Jul. 18, 2002

Related U.S. Application Data
(62) Division of application No. 09/786,015, filed on Apr. 9, 2001.
Application No. 09/786,015 is a 371 of international application No. PCT/GB99/02729, filed on Mar. 9, 2000.

(30) Foreign Application Priority Data
Aug. 28, 1998 (GB) 9818915.2

(51) Int. Cl. C12P 21/02; C07H 21/04; C12N 5/06; C07K 16/00

(52) U.S. Cl. 435/69.1; 435/326; 435/320.1; 530/388.1; 536/23.53

(57) ABSTRACT

High-affinity monoclonal antibodies, wherein the affinity is characterized by: (i) incubating first and second samples of the antibody in antigen-coated microtitre plate wells at a concentration chosen to be within the linear part of a standard curve at pH 7.2 for 1 hour at 37° C.; (ii) removing unbound antibody from both samples; (iii) incubating the first sample with PBS at pH 7.2 for 1 hour at 37° C., and reducing the pH of the second sample to pH 3 or below and incubating for 1 hour at 37° C.; (iv) removing unbound antibody from both samples; (v) incubating both samples with anti-antibody alkaline phosphatase-conjugate for 1 hour at 37° C.; (vi) removing unbound conjugate from both samples; and (vii) adding PNPP substrate to the samples, measuring the absorbance of the samples at 405 nm, and determining the amount of antibody bound to antigen, wherein the amount bound in the second sample is >50% of that of the first sample.

### FIELD OF THE INVENTION

This invention relates to antibodies and their therapeutic use.

### BACKGROUND OF THE INVENTION

Antibodies have long been regarded as potentially powerful tools in the treatment of cancer and other diseases. However, although there have been some notable exceptions, this potential has not generally yet been realised.

This relative lack of success may be due, at least in part, to the use of monoclonal antibodies derived from rodents, which seldom have affinities higher than 10⁻⁹ M. Antibodies having this level of affinity are of limited therapeutic utility, as it has proved difficult to deliver enough antibody to the target to effect useful biological activity. Antibody binding to an antigen is reversible, and at the concentrations of antibody practical for in vivo use, dissociation will be favoured over association. In principle, it is possible to counter the dissociation of antigen by increasing the antibody concentration. However, this may lead to unacceptable clinical side-effects and would also increase the costs associated with the therapy.

### SUMMARY OF THE INVENTION

The present invention is based on the realisation that antibodies, or fragments thereof, can be produced which are “acid-resistant” and that this property is associated with high affinity binding of an antibody for its antigen.

According to the present invention, a high-affinity antibody has affinity characterised by:

(i) incubating first and second samples of the antibody in antigen-coated microtitre plate wells at a concentration chosen to be within the linear response part of a standard curve at pH 7.2 for 1 hour at 37° C.;

(ii) removing unbound antibody from both samples;

(iii) incubating the first sample with PBS at pH 7.2 for 1 hour at 37° C., and reducing the pH of the second sample to pH 3 or below and incubating for 1 hour at 37° C.;

(iv) removing unbound antibody from both samples;

(v) incubating both samples with anti-antibody alkaline-phosphatase conjugate for 1 hour at 37° C.;

(vi) removing unbound conjugate from both samples; and

(vii) adding PNPP substrate to the samples, measuring absorbance of the samples at 405 nm, and determining the amount of antibody bound to antigen, wherein the amount bound in the second sample is >50% of that of the first sample.

Preferably, the maximum pH in step (iii) is 2.5, more preferably 2.0.

Antibodies or antibody fragments with the “acid-resistant” properties are expected to favour association rather than dissociation and they therefore have longer localisation times at target sites, which results in a higher concentration of antibodies localised at the target sites.

In particular, this invention relates to the production of a high affinity single-chain Fv antibody fragment. This ScFv has particular advantages in that it allows better targeting to a site in vivo.

### DESCRIPTION OF THE DRAWING

FIG. 1 illustrates the results achieved for acid-resistance of sheep and mouse monoclonal antibodies and single-chain Fvs with affinity to carcinoembryonic antigen at various pH values.

### DESCRIPTION OF THE INVENTION

The acid-resistant monoclonal antibodies according to the present invention may be obtained using various techniques. For example, classical hybridoma technology can be applied, comprising the fusion of B-lymphocytes from immunised animals secreting high-affinity antibodies with an appropriate fusion partner. An alternative method is to purify the mRNA from selected lymphocytes and use the technique of PCR to amplify the antibody genes required. Phage display technology and other techniques for the display of antibody fragments may also be used to obtain the antibody genes from naive or immunised libraries after appropriate selection procedures.

The antibody gene can be co-expressed with or otherwise chemically linked to toxins, radioisotopes or enzymes or any other desirable molecules to provide a fusion protein with strong binding characteristics. In a further alternative, the antibodies may be produced by transgenic animals as described in U.S. Pat. No. 5,770,429.

The antibody may be a whole antibody, comprising heavy and light chains, and constant and variable regions. Alternatively, the antibody is an antibody fragment, e.g. F(ab′)₂, Fab, Fv or single-chain Fv fragments, provided that at least part of the variable region is present which confers the property of “acid resistance”. The antibody may also be an animal, chimeric or humanised antibody. A suitable method for producing humanised antibodies is disclosed in WO-A-92/15699.

In a preferred embodiment of the invention, the antibody is a single-chain Fv fragment. The single-chain Fv fragment comprises both heavy chain and light chain variable regions linked by a suitable peptide.

The antibodies of the present invention may be defined by their acid-resistant properties, which can be characterised by an acid-washed enzyme-linked immunosorbent assay (EIA), as described above. Typically the A₄₀₅ value obtained by EIA will represent antibody binding of >50% for a sample at pH 3 or below, compared to the value for the sample at pH 7.2. Preferably, the A₄₀₅ value of a sample at pH 2 will represent antibody binding of >60% more preferably 70% of that obtained at pH 7.2.

The animal that is subjected to immunisation is not a rodent, but is chosen to give higher affinity antibodies. Any large mammal may be used and suitable animals include rabbits, goats, cows and sheep.

An antibody of the invention may be used in therapy and may be formulated into any suitable composition with a physiologically-acceptable excipient, diluent or carrier.

The following Examples illustrate the invention.

### EXAMPLE 1

Sheep were immunised with carcinoembryonic antigen (CEA) in complete Freund's adjuvant, then boosted three times with antigen in incomplete Freund's adjuvant. Animals were sacrificed after the final boost and lymph nodes removed.

The lymph node cells were then washed and fused with sheep heteromyeloma fusion partner SFP3.2. Fused cells were plated out at a total density of approximately 10⁶ per ml in medium containing HAT (Life Technologies). These samples were then screened for hybridomas secreting high-affinity antibodies to the specified antigen using both a normal EIA and an acid-washed EIA.

Standard EIA screening assays were carried out as follows:

Maxisorb assay plates (NUNC) were coated with CEA (0.4 μg/ml in phosphate-buffered saline at pH 7.2), 100 μl per well and left overnight at 4° C. The plates were then washed three times using phosphate buffered saline at pH 7.2 with 0.01% Tween 20 detergent. Any remaining reactive sites on the plates were blocked by the addition of 200 μl per well of 0.2% fat-free milk protein in PBS at pH 7.2 at 37° C. for ½ hour. The plates were then washed in PBS as described above and 45 μl of the antibody samples were added to the wells of the plates. The samples were incubated for one hour at 37° C. and then washed as described previously. Bound antibody was detected using alkaline phosphatase-conjugated donkey anti-sheep antibody (Sigma A5187 diluted 1/5000 in PBS at pH 7.2 with 1% BSA). The plates were then washed and 100 μl per well of PNPP (Sigma N2770) solution was added. Absorbance was measured using a spectrophotometer at 405 nm with phosphate buffered saline as a control.

Acid-wash EIA screening assays were carried out as follows:

Coating and binding of antibody samples was as described for the standard EIA above. However, after incubation with the antibody samples, the plates were washed and 200 μl per well of HCl (10 mM Stock solution) at pH 2 was added for one hour at 37° C. After three washes the antibody remaining bound to antigen was detected using alkaline phosphatase-conjugated donkey anti-sheep antibody and PNPP as described above. In order to ensure that a proper comparison was being made between antibodies at different concentrations, each sample was chosen to give an A₄₀₅ value of approximately 1.0 in the normal EIA (i.e. in the linear response part of the EIA curve).

Three hybridomas (1D2, 6G11 and 6H9) secreted antibodies which gave a greater than 50% retention of binding in the acid washed EIA, in comparison to the binding in the non-acid washed EIA.

### EXAMPLE 2

A single-chain Fv fragment was produced from the hybridoma 6H9 above, as follows:

mRNA was purified from the cultured hybridoma cells using oligo-dT cellulose. Single-stranded DNA complementary to the mRNA (cDNA) was synthesized by reverse transcription. Universal primers, designed from the constant regions of sheep heavy and light chain antibody genes, were used in separate reverse transcription reactions to synthesise the cDNA for the antibody variable regions.

The cDNA was then amplified by the polymerase chain reaction to make double-stranded DNA using primers designed from the heavy and light chain variable framework sequences. Separate polymerase chain reactions were used to amplify the heavy and light chain regions. The products were then analysed by agarose gel electrophoresis and the DNA bands equivalent to light and heavy chain genes were cut from the gel and purified.

Equimolar amounts of variable heavy and light chain DNA were mixed together with an oligonucleotide linker DNA. The linker DNA coded for the amino acid sequence (Gly₄Ser)₃ with additional nucleotides complementary to the 3′ end of the heavy chain variable region and the 5′ end of the light chain variable region. The three DNA molecules were denatured, annealed and extended in the first stage (without primers) of a two-stage PCR reaction so that the fragments were joined, thereby assembling the single-chain Fv.

The single-chain Fv DNA was amplified in the second stage of the PCR using a pair of primers derived from the heavy and light chain variable region termini with the addition of the restriction enzyme recognition sites for AlW44i and NotI. The single-chain Fv gene product was analysed by agarose gel electrophoresis and purified. The single-chain Fv was then digested with the restriction enzymes AlW44i and NotI and cloned into an expression vector. The vector was then used to transform E. coli HB 2151, and protein expression was allowed to occur. The vector was designed so as to include a hexa-histidine tag at the COOH terminus of the SFv. The single-chain Fv was purified using nickel-chelate affinity chromatography and analysed by SDS-PAGE. The amino acid sequence for the heavy chain variable region and the light chain variable region is disclosed in SEQ ID Nos. 2 and 4, respectively. An acid-wash EIA was also carried out to determine the acid-resistant properties of the single-chain Fv.

Acid-wash EIA was carried out as follows:

Carcinoembryonic antigen (CEA)-coated microtitre plates were prepared as described previously. Single-chain Fv samples (6H9) were diluted to a range of concentrations between 1 ng/ml and 100 ng/ml in PBS at pH 7.2 containing 1% bovine serum albumin (BSA). 100 μl samples were added to the microtitre plate wells and incubated for 1 hour at 37° C. The plates were then washed, 200 μl per well of citrate added, and the plates incubated for 1 hour at 37° C. In this case, the acid preparations were made using a stock solution of 100 mM citrate diluted to pH values of 4.0, 3.5, 3.0, 2.5 and 2.0 in the reaction mixture. PBS at pH 7.2 was used as a reference control. The plates were then washed and 100 μl per well of mouse anti-tetra-histidine antibody (Qiagen) (100 ng/ml diluted in PBS at pH 7.2 with 1% BSA) added and incubated for 1 hour at 37° C. After plate washing the samples were incubated for 1 hour at 37° C. With 100 μl per well of goat anti-mouse alkaline phosphatase conjugate (Sigma A3688 diluted 1/1000 in PBS with 1% BSA at pH 7.2). The plates were then washed, treated with PNPP as described previously and the absorbance measured using a spectrophotometer at 405 nm.

As a control for acid resistance, sFv samples were incubated with PBS at pH 7.2 to generate an EIA response curve for the SFv samples. In the linear region, a concentration of 10-20 ng/ml of the SFv sample gave an absorbance (A₄₀₅) of 1.0-1.5 and was therefore used to determine the amount of antibody bound in the acid washed samples as a percentage of the amount bound in the reference sample.

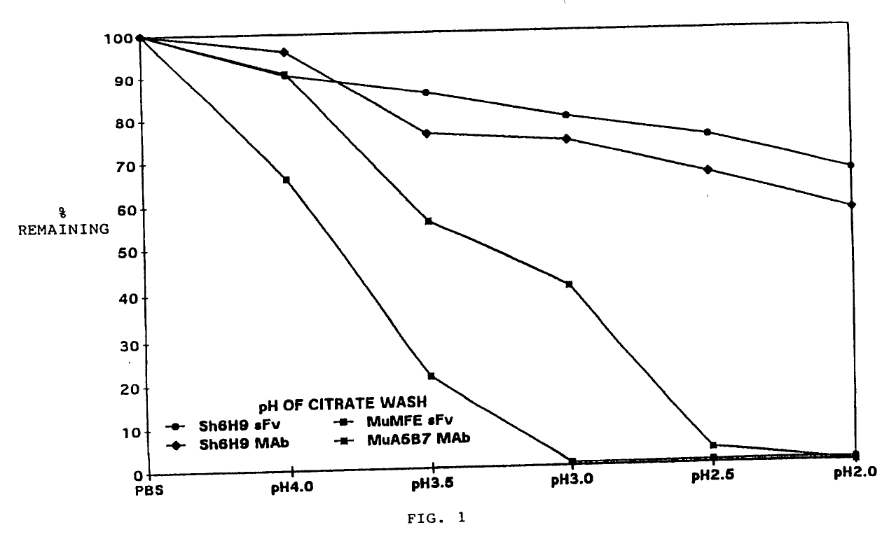

The acid-resistant properties of the 6H9 whole antibody and the 6H9 single-chain Fv were compared with that for the mouse-derived anti-carcinoembryonic antigen whole antibody, A5B7 and the single-chain Fv MFE. The results are shown in FIG. 1, with the antigen-binding of the mouse-derived antibodies being substantially reduced at pH 3.5 and less than 5% at pH 2.5. In contrast, the 6H9 antibodies retain >70% antigen at pH 3.5, >60% at pH 2.5 and >50% at pH 2.0.

I claim:

1. A polynucleotide molecule encoding a high-affinity monoclonal antibody, wherein the affinity of said antibody is characterisable by:
(i) incubating first and second samples of the antibody in antigen-coated microtitre plate wells at a concentration chosen to be within the linear part of a standard curve at pH 7.2 for 1 hour at 37° C.;
(ii) removing unbound antibody from both samples;
(iii) incubating the first sample with PBS at pH 7.2 for 1 hour at 37° C., and reducing the pH of the second sample to pH 3 or below and incubating for 1 hour at 37° C.;
(iv) removing unbound antibody from both samples;
(v) incubating both samples with anti-antibody alkaline phosphatase-conjugate for 1 hour at 37° C.;
(vi) removing unbound conjugate from both samples; and
(vii) adding PNPP substrate to the samples, measuring the absorbance of the samples at 405 nm, and determining the amount of antibody bound to antigen, wherein the amount bound in the second sample is >50% of that of the first sample,
wherein said antibody has a heavy chain variable region comprising the amino acid sequence defined in SEQ ID No. 2 and a light chain variable region comprising the amino acid sequence defined in SEQ ID No. 4, or a variant thereof;
and wherein the polynucleotide comprises a nucleotide sequence defined in SEQ ID Nos. 1 and 3, or a variant thereof.

2. A cloning vehicle comprising a polynucleotide molecule encoding a high-affinity monoclonal antibody, wherein the affinity of said antibody is characterisable by:
(i) incubating first and second samples of the antibody in antigen-coated microtitre plate wells at a concentration chosen to be within the linear part of a standard curve at pH 7.2 for 1 hour at 37° C.;
(ii) removing unbound antibody from both samples;
(iii) incubating the first sample with PBS at pH 7.2 for 1 hour at 37° C., and reducing the pH of the second sample to pH 3 or below and incubating for 1 hour at 37° C.;
(iv) removing unbound antibody from both samples;
(v) incubating both samples with anti-antibody alkaline phosphatase-conjugate for 1 hour at 37° C.;
(vi) removing unbound conjugate from both samples; and
(vii) adding PNPP substrate to the samples, measuring the absorbance of the samples at 405 nm, and determining the amount of antibody bound to antigen, wherein the amount bound in the second sample is >50% of that of the first sample;
wherein said antibody has a heavy chain variable region comprising the amino acid sequence defined in SEQ ID No. 2 and a light chain variable region comprising the amino acid sequence defined in SEQ ID No. 4, or a variant thereof;
and wherein the polynucleotide comprises a nucleotide sequence defined in SEQ ID Nos. 1 and 3, or a variant thereof.


SEQUENCE LISTING (4 sequences)

SEQ ID NO: 1 - DNA, 363 bp, Artificial Sequence
  CDS (1)..(363): Description of Artificial SequenceAntibody Fragment
caggtgcagctgcaggagtcgggacccagcctggtgaagccctcacagaccctctccctcacctgcacggtctctggattctcattaaccaagtatggtgttagttgggtccgccaggctccaggaaaggcgcttgagtggctaggtggtgtgtccagtggtgcactaacagcctataacacagccctacagtcccgactcagcgtcaccagggacacctccaagagccaattctccctgtcactgagcagcgtgactactgaggacacggccatttactactgtgcgaaatctgtcaatggtgacagtgttccttatggtttggactactggagcccaggactcctactcaccgtctcctca

SEQ ID NO: 2 - protein, 121 aa, Artificial Sequence
  Description of Artificial Sequence Antibody Fragment
QVQLQESGPSLVKPSQTLSLTCTVSGFSLTKYGVSWVRQAPGKALEWLGGVSSGALTAYNTALQSRLSVTRDTSKSQFSLSLSSVTTEDTAIYYCAKSVNGDSVPYGLDYWSPGLLLTVSS

SEQ ID NO: 3 - DNA, 333 bp, Artificial Sequence
  CDS (1)..(333): Description of Artificial sequence Antibody Fragment
caggatgtgctgactcagccgtcctccgtgtctgggtccctgggccagagggtctccatcacctgctctggaagcagcagcaacattggaggtaatgcttatgtgggctggtaccaacaggtcccaggatcagcccccagactcctcatcagtgctacaaccgatcgagcctcggggatccccgaccgattctccggctccaggtctgggaacacagccaccctgaccatcagctcgctccaggctgaggacgaggccgattattactgtgcatcgtatcaaagtacttacagtggtgttttcggcagcgggaccaggctgaccgtcctgggt

SEQ ID NO: 4 - protein, 111 aa, Artificial Sequence
  Description of Artificial Sequence Antibody
QDVLTQPSSVSGSLGQRVSITCSGSSSNIGGNAYVGWYQQVPGSAPRLLISATTDRASGIPDRFSGSRSGNTATLTISSLQAEDEADYYCASYQSTYSGVFGSGTRLTVLG